(19) United States
(12) Patent Application Publication
(10) Pub. No.: US 2023/0310633 A1
(43) Pub. Date: Oct. 5, 2023

(54) BINDING MODULATOR

(71) Applicant: Allymab, Inc., Boston, MA (US)

(72) Inventor: Jayanth Venkata Nagendra S Batchu, Charlotte, NC (US)

(21) Appl. No.: 18/012,469

(22) PCT Filed: Jun. 26, 2021
(86) PCT No.: PCT/US21/39276

§ 371 Date: Dec. 22, 2022

Related U.S. Application Data
(60) Provisional application No. 63/045,015, filed on Jun. 26, 2020.

(51) Int. Cl. A61K 47/68

(52) U.S. Cl. CPC A61K 47/6835; A61K 47/6811

(57) ABSTRACT

This invention provides a binding modulator (BMOD) system. The BMOD can bind to targeting and/or therapeutic molecule(s) (TO AT) to make a BMOD-TOAT (BMODT) complex. When the BMOD is bound to the TO AT, the BMOD can affect M various properties of the TO AT and/or BMODT complex. A first BMOD can bind to a TO AT to create a BMODT complex to affect properties of the TO AT, and one or more additional BMODs can bind to that BMODT to further modify the properties of the BMODT. This can be used to generate BMODT complex(es) with higher efficacy and functionality than the TOAT that was used to create the BMODT complexes. This higher efficacy could be a result of increased engagement with effector cells caused by the BMOD(s). Antibody biobetters can be made as well by attaching a BMOD to an antibody.

### FIELD OF THE INVENTION

This invention relates to the fields of immunology, immunotherapy, and protein engineering, and specifically to antibodies, engagers, biobetters, binding interfaces, therapies, therapeutic antibodies, improved antibodies, binding interfaces, and protein engineering.

### BACKGROUND OF THE INVENTION

Therapeutic antibodies are recognized as effective medicines which may improve overall survival time or limit disease progression in a variety of human malignancies and autoimmune conditions. The high affinity and specific binding of monoclonal antibodies has been the basis for the development of targeted immunotherapies, wherein a therapeutic molecule is conjugated to an antibody for delivery to cells expressing an antigen specifically bound by the antibody. Recombinant therapeutic antibodies have also been prepared, which incorporate effector functions of human antibody constant regions. Thus, therapeutic antibodies are effective in unlabeled or unconjugated form. For the treatment of malignancies, useful effector functions include an ability to induce antibody-dependent cellular cytotoxicity (ADCC) and an ability to activate complement. Effector functions such as inhibition of IgE are useful for treatment of autoimmune or allergy indications. Examples of therapeutic antibodies include rituximab (RITUXAN®), an anti-CD20 antibody, galiximab, an anti-CD80 antibody, and lumiliximab, an anti-CD23 antibody. RITUXAN® has demonstrated efficacy in patients with various lymphoid malignancies, including indolent and aggressive forms of B-cell non-Hodgkin's lymphoma (NHL) and B-cell chronic lymphocytic leukemia (CLL). Both galiximab and lumiliximab have shown success in clinical or preclinical trials for the treatment of B cell malignancies as well as autoimmune/allergic conditions.

The oligosaccharide component can significantly affect properties relevant to the efficacy of a therapeutic glycoprotein, including physical stability, resistance to protease attack, interactions with the immune system, pharmacokinetics, and specific biological activity. Such properties may depend not only on the presence or absence, but also on the specific structures, of oligosaccharides. Some generalizations between oligosaccharide structure and glycoprotein function can be made. For example, certain oligosaccharide structures mediate rapid clearance of the glycoprotein from the bloodstream through interactions with specific carbohydrate binding proteins, while others can be bound by antibodies and trigger undesired immune reactions. Recent studies have also shown that glycosylation contributes to antibody effector functions

There remains a continuing need to improve the efficacy of therapeutic antibodies and other therapies so that durable remissions or complete recovery from disease can be achieved, particularly in patients with relapsed or refractory disease conditions.

### SUMMARY OF THE INVENTION

This invention overcomes the disadvantages of the prior art by creating a binding modulator (BMOD) system. The BMOD can bind to targeting and/or therapeutic molecule(s) (TOAT) to make a BMOD-TAOT (BMODT) complex. Note that the terms BMODT and BMODT complex are used interchangeably herein, and the appropriate use should be clear to the reader of skill in the art. When the BMOD is bound to the TOAT, the BMOD can affect various properties of the TOAT and/or BMODT complex. Any sort of integration of a BMOD and TOAT can make a BMODT complex. A BMODT can be a biobetter, which can be an improved version of an existing biologic drug with the same target or action. The biobetter can have improved safety, efficacy, and/or ease of manufacturing, etc. A BMODT can be an improved drug and/or antibody, which can be an improved version of an existing biologic drug and/or molecule. The improved drug and/or antibody can be in development and/or can have improved safety, efficacy, and/or ease of manufacturing, etc. The BMOD can affect various properties of the BMODT complex. Said another way, a first BMOD can bind to a TOAT to create a BMODT complex to affect properties of the TOAT, and one or more additional BMODs can bind to that BMODT to further modify the properties of the BMODT. Said another way, a first BMOD can bind to a TOAT to create a BMODT complex with improved and/or different properties than that/those of the TOAT, and one or more additional BMODs can bind to that BMODT to further modify the properties of the BMODT. Each subsequent BMOD that binds to the BMODT can have further modifying effects on the properties of the BMODT. This can be used to generate BMODT complex(es) with higher efficacy and/or functionality than the TOAT that was used to create the BMODT complexes. This higher efficacy could be a result of increased engagement with effector cells caused by the BMOD. For example, a number of current antibody-based therapies can have less efficacy due to their fucosylation. This fucosylation can decrease the effectiveness of the interaction between the antibody and effector cells such as natural killer (NK) cells and cytotoxic T-lymphocytes (CTLs). A BMOD can also binds to these effector cells and if this BMOD is attached to a TOAT such as an antibody, a BMODT complex can form in which the BMODT complex can interact with effector cells via both the TOAT and the BMOD and can lead to a more effective interaction. This BMOD technology can lead to the development of new therapies and rescue older therapies. The BMOD technology can improve the efficacy and/or functionality of current therapies and/or TOATs, including in terms of cytotoxic potential and engagement potential/ability.

In an illustrative embodiment, A BMOD-TOAT (BMODT) complex is provided, and includes at least one binding modulator (BMOD), and at least one targeting and/or therapeutic agent (TOAT), wherein the at least one BMOD is bound to the at least one TOAT. The BMOD or trumodulator can be selected from the list consisting of an anti-CD3 scFv, anti-CD16 scFv, nanobody, antibody-based molecule(s), immunoglobulin based molecule(s), BiTe, BiKe, immunoglobulin, antibody, affimer, nucleotide, a synthetic lectin, immunoglobulin binding peptide, Fc binding peptide, albumin binding peptide, Fc binding ligands, FcIII-scFv fusion protein with non-canonical amino acids, FcIII based fusion protein with non-canonical amino acids, FcIII peptide with ncAA fused to a scFv, immunoglobulin based fusion molecule(s), antibody fragment, immunoglobulin fragment, antibody derivative, immunoglobulin fragment, peptides with ncAA(s), fusion proteins that include ncAA(s), binding interfaces, binding interfaces with ncAA(s), fusion protein with ncAA(s), engineered immunoglobulin based fusion molecule(s), protein A, proteins that include one or more of sequence(s) SEQ IDs 1-42 (from the listing attached hereto), peptides that include one or more of sequence(s) SEQ IDs 1-42, proteins that include one or more of sequence(s) SEQ IDs 1-42, sequences SEQ IDs 1-42, a protein with a combination of one or more of sequence(s) SEQ IDs 1-42, cytokine(s), albumin, zwitterionic amino acids, protein G, a carbohydrate binding molecule, and carbohydrate. Illustratively, the TOAT can be selected from the list consisting of a biologic, immunoglobulin, antibody, affimer, Fab fragment, immunoglobulin(s), molecule that can bind to an epitope, molecule that can bind to an antigen, molecule that can bind to an autoantigen, molecule that can specifically bind to a biologically relevant entity, ligand, IgG, antibiotic, therapy, and component of a therapy. The BMOD can be attached to the TOAT with or without a linker.

In an embodiment, a method of attaching a BMOD to a TOAT, as defined above, so as to provide a BMODT complex, incudes the steps of mixing a BMOD and a TOAT, and thereby generating, from the step of mixing, a BMODT. The step of blending the BMOD and TOAT can further comprise blending the BMOD and TOAT in a PEDIP reaction, chemical reaction, photoreactive reaction, non-radiation involved reaction, or a proximity based reaction. Also, the blending of the BMOD and TOAT in the PEDIP reaction can comprise using a chemical reaction, a photochemical reaction, or a Fc binding peptide. At least one of a scFv, affimer, aptamer, nanobody, binding interface, antibody, antibody derivative, antibody fragment, immunoglobulin, immunoglobulin derivative, immunoglobulin fragment, non-toxin, non-toxic binding interface, non-toxin binding interface, and BMOD to a TOAT or an antibody can be attached using the PEDIP reaction.

In an embodiment, a method of administering a therapy is provided, which includes at least one BMODT complex, and includes (a) collecting biological data from a patient, (b) analyzing the biological data to determine biomarker(s), (c) selecting a BMOD or engineered scFv depending on biomarker data, (d) selecting a TOAT or antibody depending on biomarker data, (e) sonding the BMOD or engineered scFv to the TOAT/antibody, and infusing/injecting the BMODT complex into the patient. The therapy involving a BMODT complex can be used to diagnose or treat cancer, autoimmune disease, rare diseases, inflammatory conditions, medical diseases, and/or medical conditions.

In an embodiment, the BMODT complex is provided such that at least one of the TOAT, trumodulator, BMODT, and BMOD binds to a solid support, semi-solid support, CD3, CD16, PD-L1, CTLA-4, PD1, NKG2D, IL-6, TNF, HER2, CD20, EGFR, IL-17R, IL-12, IL-23, VEGF, GD-2, dabigatran, CD20, BLyS, BAFF, Fc, IgG Fc region, IL-5, PCSK9, PDGFR, C. difficile toxin B, macrophages, immune cells, engineered cells, effector cells, CD33, CD123, CD22, FIXa, FX, IL-5R a subunit, CD47, checkpoint inhibitor, tumor associated antigen, tumor specific antigen, viral protein, autoantigen, microbial protein, human in vitro protein, bacterial protein, IgE, IL-12/23, P-selectin, pathogens, CD4, CD19, RSV, CD52, ITGA4, VEGF-A, C5, IL-1B, TNFa, IL-6R RANKL, BLyS, CD30, B. anthrasis PA, EGFR2, a4B7 integrin, IL-17a, CD38, SLAMF7, IL-23 p19, EpCAM, BAFF, Tau, beta amyloid, amyloid beta, Fc receptor(s), IL-4Ra, Factor IXa, Factor X, FGF23, CCR4, CGRP, IgE Fc region, Fc region, CGRPR, and von Williebrand factor. The BMODT can be a super blocker in which the TOAT is modified or attached with a BMOD that has the same or similar targeting, binding, or specificity properties as the TOAT. The BMODT complex can be a vary blocker is a BMODT complex in which the TOAT is modified or attached with a BMOD that has targeting, binding, or specificity properties for a target related to, associated with, or in the same class as the target of the TOAT. The BMODT complex can be a combo blocker in which the TOAT is modified or attached with a BMOD that has targeting, binding, or specificity properties for a target that is targeted alongside or in combination with the target of the TOAT. The BMOD can be made up of parts that can include a TOAT binder, trumodulator, anti-HAMA, rider, adsorption preventer, and deimmunizer. The BMOD can include an enhancer (EN) system that increases the performance and efficacy of a TOAT and BMODT complex when the EN system is bound to the TOAT. An immunoglobulin engager (IgENG) system can be provided with an EN system that binds to the TOAT, based upon immunoglobulin, and includes IgG and antibodies. The IgENG can be used to increase or decrease the at least one of ADCC, TDCC, ADCP, CDC, half-life, exposure, pharmacodynamic properties, pharmacokinetic properties, efficacy, functionality, and cytotoxicity of antibody-based therapies or the immunoglobulin based TOAT. The IgENG system can be used to generate the BMODT complex with increased specificity and valency when compared to the TOAT that it is based upon. A diminisher (DIM) system can be provided, and includes the BMOD. It is arranged to decrease performance and efficacy of the TOAT and the BMODT complex when the DIM system is bound to the TOAT.

### BRIEF DESCRIPTION OF THE DRAWINGS

The invention description below refers to the accompanying drawings, of which:

FIG. 1a is a schematic view showing a BMODT complex and its components, according to an illustrative embodiment;

FIG. 1b is a schematic view showing a BMODT complex in which the BMOD is connected to the TOAT with a linker, according to an illustrative embodiment;

FIG. 1c is a schematic view showing a TOAT, according to an illustrative embodiment;

FIG. 1d is a schematic view of a BMOD with possible parts, according to an illustrative embodiment;

FIG. 2a is a schematic view of an antibody-based BMODT complex with the BMOD binding to the Fc region of the antibody without using a linker, according to an illustrative embodiment;

FIG. 2b is a schematic view of an antibody-based BMODT complex with the BMOD binding to the Fab region of an antibody without using a linker, according to an illustrative embodiment;

FIG. 2c is a schematic view of an antibody-based BMODT complex with the BMOD binding near to the antigen binding site of a Fab region of an antibody and using a linker, according to an illustrative embodiment;

FIG. 2d is a schematic view of an antibody-based BMODT complex with the BMOD binding distant from the antigen binding site of a Fab region of an antibody and using a linker, according to an illustrative embodiment;

FIG. 2e is a schematic view showing an embodiment of an antibody-based BMODT complex with the BMOD binding to the Fc region of an antibody and using a linker, according to an illustrative embodiment;

FIG. 2f is a schematic view of an antibody-based BMODT complex with the BMOD binding to the Fc region of an antibody using at least one linker, according to an illustrative embodiment;

FIG. 2g is a schematic view of an antibody-based BMODT complex with the BMOD binding to a part of the Fc region of an antibody using a linker, according to an illustrative embodiment;

FIG. 3a is a schematic view of an immunoglobulin-based TOAT connected to a BMOD to form an embodiment of a BMODT complex with a linker, according to an illustrative embodiment;

FIG. 3b is a schematic view of an immunoglobulin based TOAT connected to a BMOD to form an embodiment of a BMODT complex without using a linker, according to an illustrative embodiment;

FIG. 3c is a schematic view of a BMODT complex with the BMOD binding to multiple TOAT entities using at least one linker, according to an illustrative embodiment; and

FIG. 4 is a diagram of a process for making BMODT complexes and administering BMODT complexes, according to an illustrative embodiment.

### DETAILED DESCRIPTION

This invention overcomes the disadvantages of the prior art by creating a binding modulator (BMOD) system. The BMOD can bind to targeting and/or therapeutic agent(s) (TOAT) to make a BMOD-TAOT (BMODT) complex. When the BMOD is bound to the TOAT, the BMOD can affect various properties of the TOAT and/or BMODT complex. Any sort of integration of a BMOD and TOAT can make a BMODT complex. A BMODT can be a biobetter, which can be an improved version of an existing biologic drug with the same target or action. The biobetter can have improved safety, efficacy, and/or ease of manufacturing, etc. A BMODT can be an improved drug and/or antibody, which can be an improved version of an existing biologic drug and/or molecule. The improved drug and/or antibody can be in development and/or can have improved safety, efficacy, and/or ease of manufacturing, etc. The BMOD can affect various properties of the BMODT complex. Said another way, a first BMOD can bind to a TOAT to create a BMODT complex to affect properties of the TOAT, and one or more additional BMODs can bind to that BMODT to further modify the properties of the BMODT. Said another way, a first BMOD can bind to a TOAT to create a BMODT complex with improved and/or different properties than that/those of the TOAT, and one or more additional BMODs can bind to that BMODT to further modify the properties of the BMODT. Each subsequent BMOD that binds to the BMODT can have further modifying effects on the properties of the BMODT. This can be used to generate BMODT complex(es) with higher efficacy and/or functionality than the TOAT that was used to create the BMODT complexes. This higher efficacy could be a result of increased engagement with effector cells caused by the BMOD(s). For example, a number of current antibody-based therapies can have less efficacy due to their fucosylation. This fucosylation can decrease the effectiveness of the interaction between the antibody and effector cells such as natural killer (NK) cells and cytotoxic T-lymphocytes (CTLs). A BMOD can also bind to these effector cells and if this BMOD is attached to a TOAT such as an antibody, a BMODT complex can form in which the BMODT complex can interact with effector cells via both the TOAT and the BMOD and can lead to a more effective interaction. This BMOD technology can lead to the development of new therapies and rescue older therapies. The BMOD technology can improve the efficacy and/or functionality of current therapies and/or TOATs, including in terms of cytotoxic potential and engagement potential/ability.

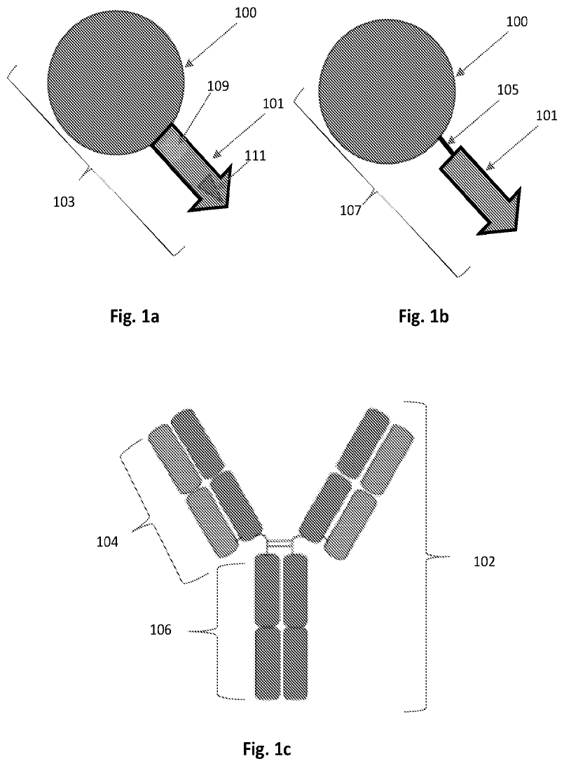

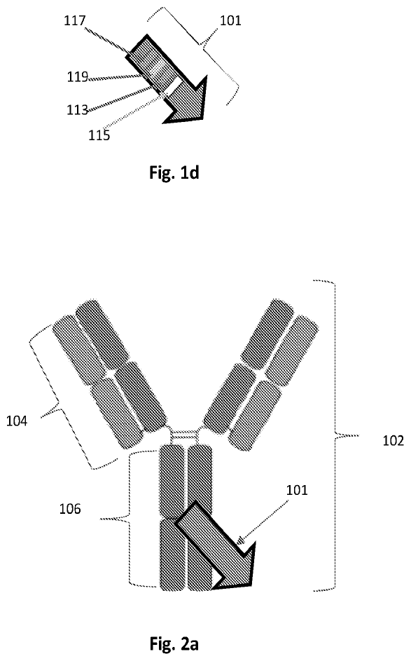

FIG. 1a is a schematic view showing a BMODT complex and its components. A targeting and/or therapeutic entity can be referred to as a TOAT 100. The binding modulator (BMOD) system can be referred to as a binding modulator, BMOD system, or as a BMOD 101. A BMOD 101 can bind to targeting and/or therapeutic TOAT(s) to make a BMOD-TAOT (BMODT) complex 103. TOAT 100 can be bound to BMOD 101 due to intermolecular interactions, intermolecular forces, and/or affinity-based interaction to form BMODT Complex 103. There can be multiple BMODs attached to a TOAT. There can be multiple TOATs attached to a BMOD. There can be multiple BMODs attached to multiple TOATs. The bond(s) between a BMOD 101 and a TOAT 100 in a BMODT complex 103 can include a chemical bond, covalent bond, photoreactive bond, high affinity interaction, biophysical interactions and/or a combination of biophysical interactions. When a BMOD 101 is bound to a TOAT 100, the BMOD 101 can affect various properties of the TOAT 100 and/or BMODT. The BMOD, once part of the BMODT complex, can still interact with the outside environment in some cases and can affect the properties of BMODT complex as a whole. For example, a BMOD could potentially cause a conformational change in the entire BMODT complex if a BMOD binds to or interacts with another molecule or atom. One or more additional BMODs can also bind to an existing BMODT complex, and the additional BMOD(s) can additionally affect the properties of the BMODT complex as a whole. The various properties being affected can include changing, modulating, controlling, or otherwise affecting the properties of the TOAT 100 and/or BMODT complex 103. Various properties being affected can include affecting the binding properties, the functionality, the efficacy, the hydrodynamic diameter, the half-life of the TOAT 100 and/or BMODT complex 103, or other various properties. A BMOD 101 can be a biomolecule, protein, nanobody, scFv, antibody-based molecule(s), immunoglobulin based molecule(s), BiTe, BiKe, immunoglobulin, antibody, affimer, affibody, nanoparticle, aptamer, affinity ligand, spiegelmer, nanobody, biocompatible molecule, molecule, nucleotide, a molecule that can bind to a biologically relevant entity, a molecular structure that can bind to a biologically relevant entity, a synthetic lectin, protein binding molecule, biomolecule binding biomolecule, immunoglobulin binding peptide, Fc binding peptide, Fc binding ligands, protein A, protein G, transferrin, hyaluronic acid, a FcIII peptide-scFv fusion protein, fusion protein, a carbohydrate binding molecule, and/or carbohydrate. A TOAT (noting that depicted TOAT 100 is one example thereof) can be a drug, molecule, molecular structure, small molecule, macromolecule, immunoglobulin, antibody, affimer, binding agent, Fab fragment, cell, immunoglobulin(s), molecule that can bind to an epitope, molecule that can bind to an antigen, molecule that can bind to an autoantigen, molecule that can specifically bind to a biologically relevant entity, antibody fragment, antibody derivative, ligand, IgG, antibiotic, therapy, and/or component of a therapy. The bond(s) and/or molecular forces between a BMOD 101 and a TOAT 100 can be created using a covalent bond, ionic bond, photoreactive bond, intermolecular forces, dipole-dipole interaction, hydrogen bond, chemical bond, intermolecular interactions, Van der Waals forces, molecular interactions, and/or affinity-based interactions. The bond(s) and/or molecular forces between a BMOD 101 and a TOAT 100 can be (including combinations and multiples of) a covalent bond, ionic bond, photoreactive bond, intermolecular forces, dipole-dipole interaction, hydrogen bond, chemical bond, intermolecular interactions, Van der Waals forces, molecular interactions, and/or affinity-based interactions. The binding properties, efficacy, cytotoxicity, hydrodynamic diameter, and/or half-life of a BMODT complex and/or TOAT can change after a BMOD is bound to a TOAT. The binding properties, hydrodynamic diameter, and/or half-life of a BMODT complex 103 and/or TOAT 100 can change after the BMOD 101 is bound to the TOAT 100. Changes in binding properties of the TOAT and or BMODT complex can include changes to affinity, changes to avidity, changes to the number of molecules the TOAT 100 can bind to, changes to which molecules the TOAT 100 can bind to, strength of the binding, affinity, strength of the binding affinity, etc. Changes in half-life can include an increase in half-life and a decrease in half-life. Changes in cytotoxicity can include an increase in cytotoxicity and a decrease in cytotoxicity. Changes in efficacy can include an increase in efficacy and a decrease in efficacy. Changes in hydrodynamic diameter can include increase in hydrodynamic diameter and a decrease in hydrodynamic diameter. A BMOD system 101 can include a TOAT binder 109 and a trumodulator 111. The TOAT binder 109 can allow and/or facilitate the binding between the BMOD system 101 and the TOAT 100. The TOAT binder 109 can be a Fc binding peptide, peptide, anti-drug antibody, molecule that can bind to a biologic drug, biointerface, affinity surface, affinity peptide, affinity molecule, Fc binding peptide with a non-canonical amino acid(s), amino acids, fusion protein, FcIII, Fc binding sequence, FcIII peptide with non-cannonical amino acid(s), Fc binding sequence with non-cannonical amino acid(s), Fc binding amino acid sequence with non-cannonical amino acid(s), Fc binding amino acid sequence, immunoglobulin binding peptide, protein A, and/or affinity ligand. The trumodulator 111 can affect various properties including the binding properties, functionality, efficacy, specificity, selectivity, hydrodynamic diameter, and/or half-life of the TOAT 100 and/or the BMODT complex 103. Affecting the various properties of the TOAT 100 and/or the BMODT 103 can include changing, modulating, controlling, and/or otherwise affecting the properties. A TOAT binder 109 can be chemically linked to a trumodulator 111. A TOAT binder 109 can be fused to a trumodulator 111. A BMOD 101 can be a fusion protein and/or fusion molecule that includes a TOAT binder 109 and/or trumodulator 111. A BMOD 101 can include a trumodulator 111 with a label and/or tag that can include a non-canonical amino acid(s). A BMOD 101 can be a fusion protein and/or a fusion molecule that can include a TOAT binder 109, a trumodulator 111, and/or an engineered/modified version of a trumodulator 111. A BMOD 101 can include a trumodulator 111 and/or a TOAT binder 109 that can include a non-canonical amino acid(s). A TOAT 100 and/or BMOD 101 can be made with peptide synthesis, protein synthesis, translation of a genetic construct, and/or other means of synthesis/construction. A BMOD 101 can be made without using conjugation chemistry and/or conjugating two separate molecular structures. In an embodiment, a trumodulator 111 can be bound to a TOAT binder 109 without having to be conjugated and/or having to use conjugation chemistry. A trumodulator 111 engineered to include a TOAT binder 109 can be a BMOD 101. A BMOD 101 can include a trumodulator 111 and/or a TOAT binder 109. A BMOD 101 can be a fusion protein or fusion molecule that includes a trumodulator 111 and/or a TOAT binder 109. In an embodiment, a TOAT binder 109 can be bound to a trumodulator 111 with a peptide bond. A PEptide-DIrected Photo-cross-linking (PEDIP) reaction can be used to conjugate, bond, link, and/or connect a BMOD 101 to a TOAT 100. A BMOD and a TOAT can be mixed together and exposed to radiation to conjugate, bond, link, and/or connect them together and make a BMODT complex. As described in additional detail below, a trumodulator 111 can be (including combinations and multiples of) a biomolecule, protein, nanobody, scFv, antibody-based molecule(s), immunoglobulin based molecule(s), BiTe, BiKe, immunoglobulin, antibody, affimer, affibody, nanoparticle, aptamer, antibody derivative, antibody fragment, binding interface, affinity ligand, spiegelmer, nanobody, biocompatible molecule, molecule, nucleotide, a molecule that can bind to a biologically relevant entity, a molecular structure that can bind to a biologically relevant entity, a synthetic lectin, protein binding molecule, biomolecule binding biomolecule, immunoglobulin binding peptide, Fc binding peptide, Fc binding ligands, protein A, protein G, transferrin, hyaluronic acid, a FcIII peptide-scFv fusion protein, fusion protein, a carbohydrate binding molecule, and/or carbohydrate.

An enhancer (EN) system can be a BMOD system 101 that can increase the performance and/or efficacy of a TOAT 100 and/or BMODT complex 103 when the EN system is bound to the TOAT 100. An enhancer system can be referred to as an enhancer or an EN system. In an embodiment of an EN system, the trumodulator 111 can be used to increase the efficacy of the TOAT 100 and/or BMODT complex 103. In an embodiment, the trumodulator 111 of an EN system can be a performance and/or efficacy molecule(s) (PERFEFF). In an embodiment, the trumodulator 111 of an EN system can target and/or bind to a PERFEFF. A PERFEFF can be a molecule that can increase the efficacy and/or performance of a BMOD system 101, TOAT 100, and/or BMODT complex 103. A PERFEFF can be a molecule that can increase the efficacy and/or performance of a BMOD system 101, TOAT 100, and/or BMODT complex 103 when targeted and/or bound. A PERFEFF can be any molecule(s) that can trigger a cytotoxic response, any entity that can contribute to triggering and/or modulating a cytotoxic response, a desired response, a triggered response, a biomolecule indicative of a target, a biomolecule indicative of a target cell, and/or any entity that can contribute to a cytotoxic response. Examples of PERFEFFs can include molecules that target and/or bind to CD16, PD-1, PD-L1, CTLA-4, and CD3. In an embodiment, the trumodulator 111 can be a molecule or a derivative of that molecule, in which a molecule can be a scFv, affimer, Fab region, immunoglobulin, binding interface, nanobody, molecule that can bind to an epitope, molecule, TOAT fusion protein, TOAT fusion molecule, molecule that can specifically bind to a biologically relevant entity, aptamer, affibody, TOAT, drug, transferrin, hyaluronic acid, synthetic lectin, peptide, FcIII peptide-scFv fusion protein, FcIII peptide-scFv fusion protein with non-canonical amino acids, fusion protein, antibody, spiegelmer, polyethylene glycol (PEG), etc. In an embodiment, trumodulator 111 can be a TOAT. In an embodiment, a TOAT can be trumodulator 111. In various embodiments, the agents that can be or can be used to make a TOAT can also be or be used to make a trumodulator. In an embodiment, an EN system can be used to bind to a TOAT to make a BMODT complex that has a larger hydrodynamic diameter and/or half-life than those/that of a TOAT. In an embodiment, the trumodulator of an EN system can increase or maximize the targeting and/or binding to a PERFEFF. In an embodiment, the trumodulator of an EN system can minimize, reduce, or avoid targeting and/or binding to an anti-performance and/or efficacy molecule(s) (APERFEFF). An APERFEFF can decrease the efficacy and/or performance of a TOAT and/or BMODT complex when targeted and/or bound. An APERFEFF can be a target molecule to avoid and/or target molecule. An APERFEFF can be a trumodulator 111 in an embodiment of a BMOD and/or BMODT complex.

A diminisher (DIM) system can be a BMOD system 101 that can decrease the performance and/or efficacy of a TOAT 100 and/or BMODT complex 103 when the DIM system is bound to the TOAT 100. A diminisher system can be referred to as a diminisher. A diminisher system can be used to mitigate and/or reduce toxicity of TOATs, including, afucosylated antibodies, antibodies, immunoglobulins, etc. In an embodiment of a DIM system, the trumodulator 111 can be used to decrease the performance and/or efficacy of the TOAT 100 and/or BMODT complex 103. In an embodiment, the trumodulator 111 of a DIM system can be an APERFEFF. In an embodiment, the trumodulator 111 of a DIM system can target and/or bind to an APERFEFF. In an embodiment, the trumodulator 111 of a DIM system can target and/or bind to nothing and/or no biologically relevant molecule. In an embodiment, the trumodulator is not meant to bind to anything in particular even though it may interact with other molecules (in its environment) in passing. This can be used to extend or affect half-life properties. In an embodiment, the trumodulator 111 of a DIM system can minimize, reduce, or avoid targeting and/or binding to a PERFEFF. In an embodiment, the trumodulator 111 of a DIM system can increase or maximize the targeting and/or binding to an APERFEFF entity. In an embodiment, the trumodulator 111 of a DIM system can decrease or minimize targeting and/or binding to a PERFEFF. Reference is made to the attached sequence listings, entitled SEQ ID 1-SEQ ID 42. In various embodiments, the trumodulator 111 can be (including combinations and multiples of) a scFv, nanobody, affimer, Fab region, immunoglobulin, a molecule that can bind to an epitope, a molecule that can specifically bind to a biologically relevant entity, an aptamer, a speigelmer, albumin, peptide, albumin binding peptide (such as SEQ ID 40), FcRn binding peptide (such as SEQ IDs 36-39), anti-CD16 scFv (such as SEQ ID 42), anti-CD3 scFv (such as SEQ ID 41), antibody, autoantigen, autoantigen fragments, antibody, antibody fragment, antibody derivative, immunoglobulin, immunoglobulin fragment, immunoglobulin derivative, affibody, biologic drug, FcIII peptide fused to a scFv, FcIII peptide with a ncAA(s) fused to a scFv (such as SEQ IDs 1-2, 4-11, 16-28, and 35), FcIII peptide with a ncAA(s) fused to a peptide/protein (such as SEQ IDs 3, 12-15, and 29-34), affinity ligand, amino acids, glycine, serine, Gly3Ser repeats, combinations of glycine and serine, Gly4Ser repeats, PEG, etc. In various embodiments, the trumodulator 111 can be created using a scFv, nanobody, affimer, Fab region, immunoglobulin, a molecule that can bind to an epitope, a molecule that can specifically bind to a biologically relevant entity, an aptamer, a speigelmer, albumin, binding interface, amino acids, Gly4Ser repeats, PEG, etc. In an embodiment, a DIM system can be used to bind to a TOAT 100 to make a BMODT complex 103 that has a larger hydrodynamic diameter than that of the TOAT 100. In an embodiment, a DIM system can be used to bind to a TOAT 100 to make a BMODT complex 103 that has a larger half-life than that of the TOAT 100. Various sequence listings applicable to the above-referenced compounds are appended hereto by way of non-limiting example. In various embodiments, the BMOD 101 can be (including combinations and multiples of) a scFv, nanobody, affimer, Fab region, immunoglobulin, a molecule that can bind to an epitope, a molecule that can specifically bind to a biologically relevant entity, an aptamer, a speigelmer, albumin, peptide, albumin binding peptide (such as SEQ ID 40), FcRn binding peptide (such as SEQ IDs 36-39), anti-CD16 scFv (such as SEQ ID 42), anti-CD3 scFv (such as SEQ ID 41), antibody, autoantigen, autoantigen fragments, antibody, antibody fragment, antibody derivative, immunoglobulin, immunoglobulin fragment, immunoglobulin derivative, affibody, biologic drug, FcIII peptide fused to a scFv, FcIII peptide with a ncAA(s) fused to a scFv (such as SEQ IDs 1-2, 4-11, 16-28, and 35), FcIII peptide with a ncAA(s) fused to a peptide/protein (such as SEQ IDs 3, 12-15, and 29-34), affinity ligand, amino acids, glycine, Gly3Ser repeats, combinations of glycine and serine, Gly4Ser repeats, PEG, etc.

In an embodiment, the trumodulator 111 in a BMOD system 101 can be a molecule that is a sensor and/or a molecule that can be sensed and/or can be applied for diagnostic and/or continuous monitoring purposes.

FIG. 1b is a schematic view showing a BMODT complex in which the BMOD is connected to the TOAT with a linker. TOAT 100 can be bound to BMOD 101 with a linker 105 to form BMODT complex 107. Linker 105 can be (including combinations and multiples of) a covalent bond, ionic bond, photoreactive bond, intermolecular forces, dipole-dipole interaction, hydrogen bond, chemical bond, covalent bond, ionic bond, intermolecular interactions, nanoparticle(s), Van der Waals forces, molecular interactions, glycine, serine, Gly3Ser repeats, combinations of glycine and serine, Gly4Ser repeats, intermolecular forces, serine, glycine, amino acids, PEptide-DIrected Photo-cross-linking (PEDIP) reaction, synthetic amino acids, non-proteinogenic amino acids, affinity-based interactions, crosslink, hydrogen bonds, protein-protein interfaces, molecular interfaces, and/or affinity-based interactions. Linker 105 can be created using a covalent bond, ionic bond, photoreactive bond, intermolecular forces, dipole-dipole interaction, hydrogen bond, chemical bond, covalent bond, ionic bond, intermolecular interactions, nanoparticle(s), Van der Waals forces, molecular interactions, intermolecular forces, serine, crosslinker, glycine, amino acids, PEptide-DIrected Photo-cross-linking (PEDIP) reaction, synthetic amino acids, non-proteinogenic amino acids, affinity-based interactions, hydrogen bonds, protein-protein interfaces, molecular interfaces, and/or affinity-based interactions. BMODT Complex 103 can have very similar and/or the same properties as BMODT Complex 107. There can be multiple BMODs attached to a TOAT. There can be multiple TOATs attached to a BMOD. There can be multiple BMODs attached to multiple TOATs. Turning to FIGS. 1a and 1b, BMODT complex 103 can be a general form (with or without a linker) of a BMODT complex and BMODT complex 107 can be a specific version or embodiment of the general form that has a linker. A linker 105 can improve the stability of the BMODT complex and the overall performance. A linker 105 can decrease the risk of the breakdown of a BMODT complex. A linker 105 can improve the pharmacodynamics, biodistribution, stability, and biocompatibility of a BMODT complex. The reduced risk of breakdown can be very important in the storage, administration, safety, and performance of BMODT complexes. A linker 105 can be at least one bond long. A BMOD 101 can include a trumodulator 111, linker 105, and/or TOAT binder 109 that can include a non-canonical amino acid(s). A BMOD 101 can include a trumodulator 111, linker 105, non-canonical amino acid(s), and/or TOAT binder 109 that can include a non-canonical amino acid(s). A BMOD 101 can be used in a radiation-induced site-specific conjugation of a TOAT 100. A radiation-induced site-specific conjugation can be used to bind a BMOD 101 to a TOAT 100. A BMOD 101 can be crosslinked to a TOAT 100. In an embodiment, only a single conjugation can be used to make a BMODT complex. In an embodiment, the only conjugation used to make a BMODT complex can be conjugating a BMOD to a TOAT. In an embodiment, the only conjugation used to make a BMODT complex can be conjugating a BMOD to a TOAT using the PEDIP reaction. In an embodiment, a proximity-induced site-specific conjugation can be used to bind a BMOD 101 to a TOAT 100 in a manner that should be clear to those of skill. In an embodiment, a non-radiation-induced site-specific conjugation can be used to bind a BMOD 101 to a TOAT 100 in a manner that should be clear to those of skill. A BMOD 101 bound to a TOAT 100 without a linker can make a BMODT complex in which intermolecular forces can contribute more to bond strength between a BMOD 101 and a TOAT 100 than intramolecular forces contribute. A BMOD 101 bound to a TOAT 100 with a linker such as linker 105 can make a BMODT complex in which intermolecular forces can contribute less to bond strength between a BMOD 101 and a TOAT 100 than intramolecular forces contribute.

The BMOD 101, BMODT complex 103, and/or BMODT complex 107 can be of low immunogenicity, biocompatible, and/or biodegradable. The BMOD 101, BMODT complex 103, and/or BMODT complex 107 can be humanized, be engineered to have reduced immunogenicity, and/or be engineered to produce the desired response. An embodiment of the BMOD 101 can be used to make a BMODT complex of a certain size and/or hydrodynamic diameter that can fit and/or function in an immunological synapse. The BMOD 101, BMODT complex 103, and/or BMODT complex 107 can be subject to the FcRn antibody recycling pathway. The affinity and/or binding properties of the BMOD 101, BMODT complex 103, and/or BMODT complex 107 to FcRn can be modified and/or optimized through biomedical and molecular engineering approaches. This modification/modulation can change half-life properties and/or the amount of time BMOD 101, BMODT complex 103, and/or BMODT complex 107 spend in serum. The pH properties, including elution pH, of the BMOD 101, BMODT complex 103, and/or BMODT complex 107 can be modified so that the BMOD 101, BMODT complex 103, and/or BMODT complex 107 can perform their intended functions. The elution pH of the BMOD 101, BMODT complex 103, and/or BMODT complex 107 can be engineered/modified so that it cannot be in physiologically relevant pH(s) or so that the BMOD 101, BMODT complex 103, and/or BMODT complex 107 don't get disassembled or unbound/eluted from a TOAT (such as TOAT 100), APERFEFF entity, and/or PERFEFF entity. This is because in a certain situation(s) the BMOD can be unbound from the TOAT, thereby breaking the BMODT. The elution pH of the BMOD 101, BMODT complex 103, and/or BMODT complex 107 can be engineered/modified so that it can be in physiologically relevant pH(s) or so that the BMOD 101, BMODT complex 103, and/or BMODT complex 107 get disassembled or unbound/eluted from a TOAT (such as TOAT 100), APERFEFF entity, and/or PERFEFF entity. Using the BMOD 101, BMODT complex 103, and/or BMODT complex 107 can result in or lead to a lower dose of a TOAT for a therapy and/or can be used to treat more patients with a lower amount of TOAT because one unit of a BMOD or BMODT complex based therapy can be more efficacious than one unit of just a TOAT based therapy.

The BMOD 101 can be used to bind to a TOAT 100 to make a BMODT complex 103 and/or BMODT complex 107 that can be or act like a cell engager, including acting like a T-cell engager. For example, if the TOAT 100 is an anti-EpCAM antibody and a BMOD 101 containing an anti-CD3 scFv was used to bind the anti-EpCAM antibody to form a BMODT complex 103 and/or BMODT complex 107, it could become a bispecific antibody that is functionally similar to Catumaxomab. It could also bind and/or engage T-cells.

FIG. 1c is a schematic view showing a TOAT. Antibody 102 is an example of a TOAT. Fab region 104 (depicted with lighter shading in the antibody herein) and Fc region 106 are parts of antibody 102. Other examples of TOATs can include a Fc region, peptibody, Fc-fusion protein, fusion proteins, etc. a TOAT can include abatacept, benlysta, rituximab, trastuzumab, etc.

FIG. 1d is a schematic view showing a BMOD and parts that can be included in a BMOD. The BMOD 101 can further include an anti-HAMA 117, rider 119, an adsorption preventer 113, and/or a deimmunizer 115. An anti-HAMA 117 can be a molecule that binds to human anti-mouse antibodies (HAMA) and/or prevents/inhibits HAMA from binding to a BMOD and/or BMODT complex. The rider 119 can be a molecule that can bind to a molecule such as an environmental entity to increase half-life of a BMOD and/or BMODT complex. An environmental entity can be a molecule that can be found in an environment the BMOD and/or BMODT complex are in. An example of an environmental entity can be albumin. In an embodiment, rider 119 can be an albumin binding peptide. In an embodiment, rider 119 can be a sequence of amino acids. An adsorption preventor 113 can be a molecule, amino acid(s), including zwitterionic molecules, that can prevent/inhibit the formation of a protein corona for a BMOD and/or BMODT complex and/or decrease/minimize the hydrodynamic diameter of a BMOD and/or BMODT complex. In an embodiment, another molecule can be conjugated to an adsorption preventor 113. In an embodiment, another molecule can be conjugated to an adsorption preventor 113 and not conjugated to a TOAT binder 109. In an embodiment, a trumodulator 1111 can be conjugated to an adsorption preventor 113 and not conjugated to a TOAT binder 109. In an embodiment, a BMOD 101 can include a TOAT binder 109, adsorption preventor 113, and/or trumodulator 111 in which a trumodulator 111 can be conjugated to an adsorption preventor 113. In an embodiment, a BMOD 101 can include a TOAT binder 109, adsorption preventor 113, and/or trumodulator 111 in which a trumodulator 111 can be conjugated to an adsorption preventor 113 and not conjugated to a TOAT binder 109. A deimmunizer 115 can be a molecule that can reduce the immunogenicity of a BMOD and/or BMODT complex. A deimmunizer 115 can be a PEG attachment/coating, CD47 molecule based attachment/coating, sequence of amino acids, Gly3Ser repeats, Gly4Ser repeats, repeats of amin acids, and/or PD-L1 molecule based attachment/coating. The BMOD 101 can further include a part that can also modify the BMOD, affect other properties of the BMOD and BMODT complexes, and/or impart and/or detract other properties of the BMOD and BMODT complex as well.

A part of the BMOD can potentially function as other part(s) of the BMOD. In an embodiment, one part can have more than one function and can fulfill the functions of other part(s) as well. In an embodiment, the trumodulator can include an anti-HAMA 117, rider 119, an adsorption preventor 113, and/or a deimmunizer 115. In an embodiment, the trumodulator can have the properties of multiple parts.

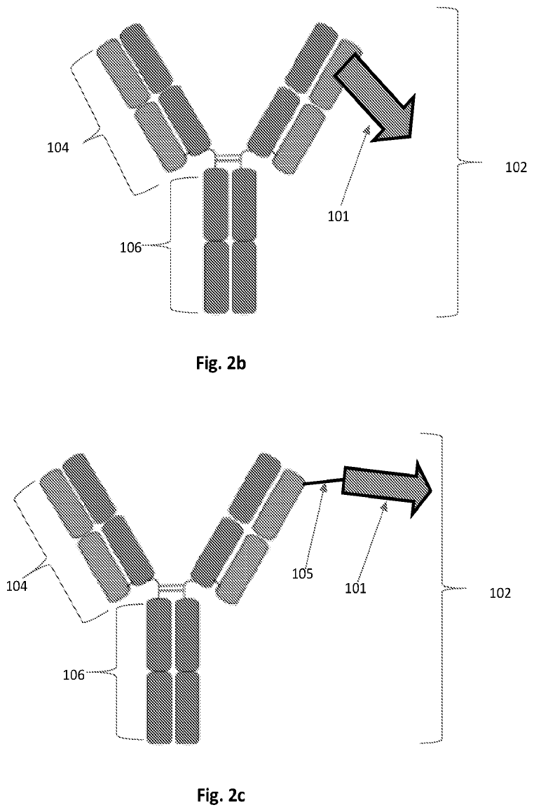

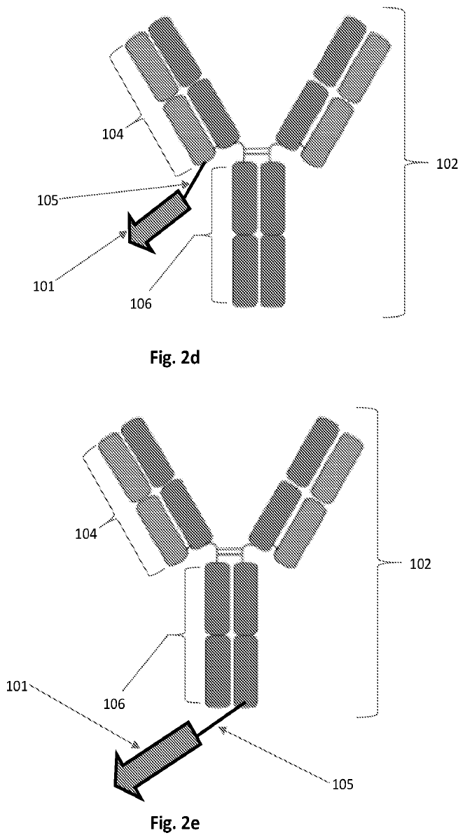

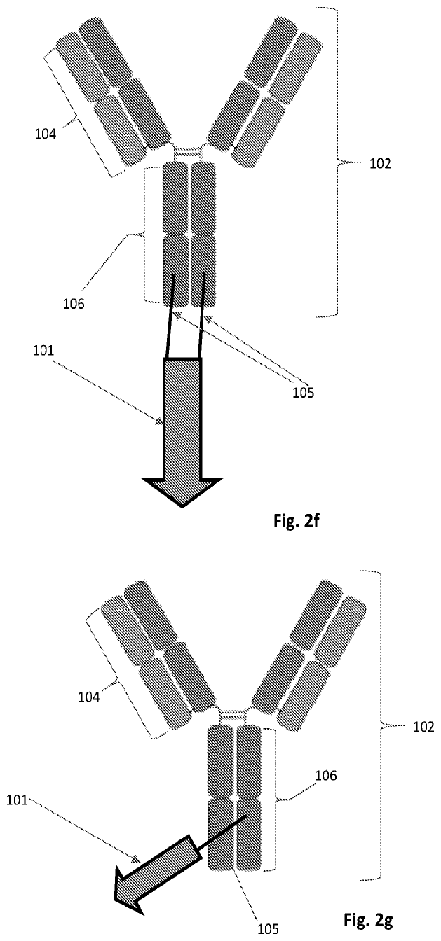

FIG. 2a is a schematic view showing an embodiment of an antibody-based BMODT complex with the BMOD binding to the Fc region of the antibody without using a linker such as linker 105. BMOD 101 is binding to antibody 102 on the antibody's Fc region 106. BMOD 101 can bind to antibody 102 in this manner using intermolecular forces, affinity-based interactions, hydrogen bonds, protein-protein interfaces, and/or molecular interfaces. In an embodiment, an immunoglobulin engager (IgENG) system can be an EN system that binds to an immunoglobulin based TOAT, including IgG and antibody 102, with or without a linker 105 and/or a covalent bond. The IgENG system can also be referred to as IgENG. An IgENG can be used to increase the ADCC, T-cell directed cell cytotoxicity (TDCC), antibody dependent cell-mediated phagocytosis (ADCP), complement dependent cytotoxicity (CDC), half-life, exposure, efficacy, functionality, and/or cytotoxicity of antibody-based therapies. An IgENG can be used to decrease the ADCC, TDCC, ADCP, CDC, half-life, exposure, efficacy, functionality, and/or cytotoxicity of antibody-based therapies. An IgENG system can be used to make immunoglobulin-based molecules bispecific, trispecific, quadraspecific, bivalent, trivalent, tetravalent, multivalent, and/or multispecific. This can significantly increase the efficacy of immunoglobulin-based therapies.

FIG. 2b is a schematic view showing an embodiment of an antibody-based BMODT complex with the BMOD binding to the Fab region of an antibody without using a linker. BMOD 101 is binding to antibody 102 on the antibody's Fab region 104. Binding of a BMOD to the Fc and/or Fab regions can lead to BMODT complexes with different efficacies and performance. This can be because binding to certain parts of the antibody can affect the affinity of its binding interfaces. Overall avidity of a BMODT complex may be affected as well.

FIG. 2c is a schematic view showing an embodiment of an antibody-based BMODT complex with the BMOD binding near to the antigen binding site of a Fab region of an antibody using a linker. BMOD 101 is binding to antibody 102 on the antibody's Fab region 104 using linker 105. Linker 105 can be or can be created using a covalent bond, ionic bond, photoreactive bond, intermolecular forces, dipole-dipole interaction, hydrogen bond, chemical bond, covalent bond, ionic bond, intermolecular interactions, Van der Waals forces, molecular interactions, intermolecular forces, affinity-based interactions, serine, glycine, amino acids, synthetic amino acids, non-proteinogenic amino acids, nanoparticle(s), PEptide-DIrected Photo-cross-linking (PEDIP) reaction(s), photoreactive reaction(s), site-specific modification(s), site-specific reaction(s), hydrogen bonds, protein-protein interfaces, molecular interfaces, and/or affinity-based interactions. In an embodiment, multiple linkers 105 can be linking/between a BMOD(s) and a TOAT(s). In an embodiment, linker 105 is attaching BMOD 101 to Fab region 104 at the n-terminus. Binding of a BMOD to the Fc and/or Fab regions, with or without a linker, can lead to BMODT complexes with different efficacies and performance. This can be because binding to certain parts of the antibody can affect the affinity of its binding interfaces.

FIG. 2d is a schematic view showing an embodiment of an antibody-based BMODT complex with the BMOD binding not near the antigen binding site of a Fab region of an antibody using a linker. BMOD 101 is binding to antibody 102 on the antibody's Fab region 104 using linker 105. In an embodiment, linker 105 is attaching BMOD 101 to Fab region 104 at the c-terminus. Binding of a BMOD to the Fc and/or Fab regions can lead to BMODT complexes with different efficacies and performance. This can be because binding to certain parts of the antibody can affect the affinity of its binding interfaces.

FIG. 2e is a schematic view showing an embodiment of an antibody-based BMODT complex with the BMOD binding to the Fc region of an antibody using a linker. BMOD 101 is binding to antibody 102 on the antibody's Fc region 106 using linker 105. In an embodiment, linker 105 is attaching BMOD 101 to Fc region 106 at the c-terminus. In an embodiment, a BMOD and/or BMODT complex can be a TOAT. In an embodiment, the agents that can be or can be used to make a TOAT can also be or be used to make a BMOD and/or BMODT complex. In an embodiment, a BMOD and/or BMODT complex can be an engineered and/or labeled TOAT. The BMODT complex can be multiple TOATs linked together. Binding of a BMOD to the Fc and/or Fab regions can lead to BMODT complexes with different efficacies and performance. This can be because binding to certain parts of the antibody can affect the affinity of its binding interfaces. Overall avidity of a BMODT complex can be affected as well.

FIG. 2f is a schematic view showing an embodiment of an antibody-based BMODT complex with the BMOD binding to or part of the Fc region of an antibody using at least one linker or with the BMOD within a TOAT. BMOD 101 is binding to antibody 102 on the antibody's Fc region 106 using multiple linkers 105. Each linker can be a different embodiment of linker 105. For example, one unit can be a covalent bond and another linker can be an intermolecular forces based interaction/bond. BMOD 101 can be attached to antibody 102 in multiple ways. Another example is that the BMOD can be inserted into the linker region of a bispecific T-cell engager to create a BMODT complex.

FIG. 2g is a schematic view showing an embodiment of an antibody-based BMODT complex with the BMOD binding to a part of the Fc region of an antibody using a linker. BMOD 101 is binding to antibody 102 on the antibody's Fc region 106 using linker 105. In an embodiment, BMOD 101 can include molecules that activate and/or bind to CD16, CD3, and/or Fc region. Linker 105 can be a combination of a Fc binding peptide (with or without non-canonical amino acid(s)) and a covalent bond (including those made through photoreactive means). The Fc binding peptide, including those that can be chemically reactive and/or photoreactive, portion of the linker can be used to bind to Fc region 106 of antibody 102 and subsequently react chemically with antibody 102 and/or photoreactively interact with antibody 102 to form a bond, such as a chemical bond and/or a covalent bond. In an embodiment, the Fc binding peptide can react based on proximity. In an embodiment, a DIM system could be used such that the BMOD can bind to (the functional) parts of a Fc region so as to decrease the function of the Fc region. In an embodiment, this can be used to create a BMODT complex that can include a DIM system in which the TOAT can be Catumaxomab. In an embodiment, a BMODT complex based on Catumaxomab could have lower side effects and/or deliver therapeutic benefit. In an embodiment, this can be due to a reduced interaction of certain white blood cells such as Kupffer cells. In an embodiment, a BMODT complex based on a DIM system can be used to render a Fc region non-functional. In an embodiment, an immunoglobulin engager (IgENG) system can be an EN system that binds to an immunoglobulin based TOAT, including IgG and antibody 102, with or without a linker 105 and/or a covalent bond. The IgENG system can also be referred to as IgENG. An IgENG can be used to increase the ADCC, T-cell directed cell cytotoxicity (TDCC), antibody dependent cell-mediated phagocytosis (ADCP), complement dependent cytotoxicity (CDC), half-life, exposure, efficacy, functionality, and/or cytotoxicity of antibody-based therapies. An IgENG can be used to decrease the ADCC, TDCC, ADCP, CDC, half-life, exposure, efficacy, functionality, and/or cytotoxicity of antibody-based therapies. An IgENG system can be used to make immunoglobulin-based molecules bispecific, trispecific, quadraspecific, bivalent, trivalent, tetravalent, multivalent, and/or multispecific. This can significantly increase the efficacy of immunoglobulin-based therapies.

A scFv can be added to an antibody using the PEDIP reaction. Adding a BMOD such as a scFv to a TOAT, such as an antibody, using the PEDIP reaction can have many advantages. This can allow for a BMODT complex involving an antibody to be manufactured in a simpler and/or easier way. Adding a BMOD such as a scFv to an antibody can let the modified antibody be a NK cell engager, T cell engager, macrophage engager, monocyte engager, etc. The scFv can be binding to a NK cell, T-cell, macrophage, monocyte, etc. to engage them. Adding a BMOD such as a scFv to an antibody can let the modified antibody be a “super blocker.” A super blocker can be a BMODT complex in which the TOAT, biologic, and/or antibody can be modified/attached with a BMOD that has the same or similar targeting, binding, and/or specificity properties as the antibody/TOAT. For example, a super blocker can be an anti-PD-1 antibody attached with a BMOD(s) that can also bind to PD-1. Adding a BMOD such as a scFv to an antibody can let the modified antibody be a “vary blocker” or “variblocker.” A vary blocker can be a BMODT complex in which the TOAT, biologic, and/or antibody can be modified/attached with a BMOD that has targeting, binding, and/or specificity properties for a target related or associated (or in the same class as) with the target of the antibody/TOAT. For example, a vary blocker can be an anti-PD-1 antibody attached with a BMOD(s) that can bind to another checkpoint inhibitor such as CTLA-4. Adding a BMOD such as a scFv to an antibody can let the modified antibody be a “combo targetter” or “combo blocker.” A combo targetter or combo blocker can be a BMODT complex in which the TOAT, biologic, and/or antibody can be modified/attached with a BMOD that has targeting, binding, and/or specificity properties for a target that can be targeted alongside and/or in combination with the target of the antibody/TOAT. A combo blocker can be used to simplify combination therapies and/or have a combo blocker target (or co-target) the same combination of targets as those of the combination therapy. The synergy of combination therapy can be delivered with a combo blocker. In an embodiment, a BMOD can be a Fc fragment or Fc region. In an embodiment, a BMOD can be albumin, part of an albumin molecule, a molecule that can bind to albumin, and/or polyethylene glycol (PEG). In an embodiment, there can be one or two or more BMODs bound to a TOAT to make a BMODT complex.

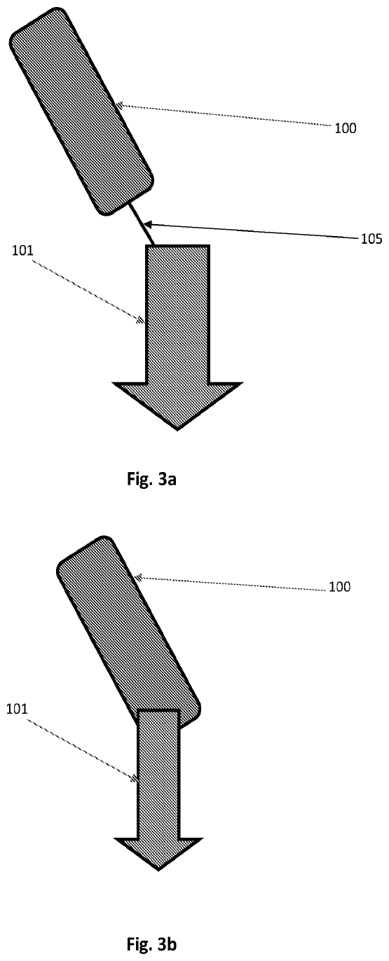

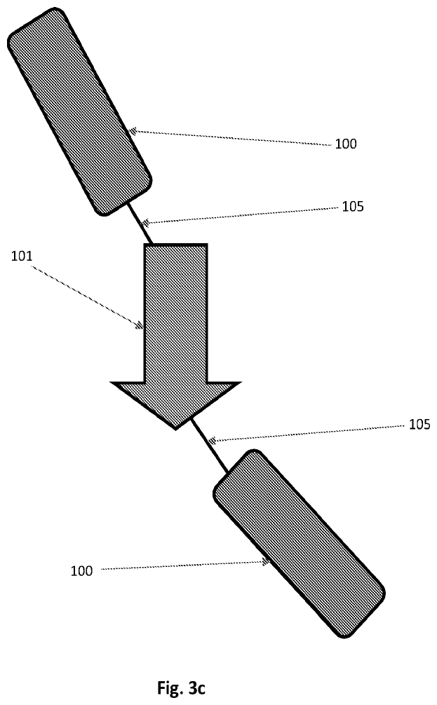

FIG. 3a is a schematic view showing an embodiment of an immunoglobulin based TOAT connected to a BMOD to form an embodiment of a BMODT complex with a linker. TOAT 100 is bound to BMOD 101 using linker 105. TOAT 100 can include scFv(s), BiTe(s), immunoglobulin-based molecules, molecules with binding properties, etc.

FIG. 3b is a schematic view showing an embodiment of an immunoglobulin based TOAT connected to a BMOD to form an embodiment of a BMODT complex without using a linker. TOAT 100 is bound to BMOD 101. TOAT 100 can be bound to BMOD 101 using intermolecular forces, protein interfaces, biomolecule interfaces, and/or high affinity interactions.

FIG. 3c is a schematic view showing an embodiment of a BMODT complex with the BMOD binding to multiple TOAT entities using at least one linker or with the BMOD within a TOAT. A BMOD can be embedded within a TOAT or multiple TOATs. In an embodiment, BMOD 101 is connected to a TOAT 100 using linker 105 and another TOAT 100 using linker 105. In an embodiment, BMOD 101 can be connected to a TOAT 100 using linker 105 and another, different TOAT 100 using linker 105. In an embodiment, BMOD 101 can be embedded between parts of a TOAT 100 and/or within a TOAT 100 and can be connected to the (parts of) TOAT 100 using linker(s) 105. In an embodiment, the Fc region of an antibody can be replaced by a BMOD. In an embodiment, a BMOD can connect multiple Fab regions. In an embodiment, a BMOD can connect and/or link components of a BiTe.

An example sequence and/or general structure for a BMODT complex can be TOAT sequence/region-linker sequence/region-BMOD sequence/region. Specific examples can include trastuzumab-Fc binding peptide with a photoreactive part-scFv against CD16 and trastuzumab-Fc binding peptide with a photoreactive part-scFv against CD3. In an embodiment, non-canonical amino acids or ncAA(s) include photoreactive amino acids and photoreactive amino acid analogue(s), including p-benzoylphenylalanine (pBpa). In an embodiment, the BMOD or trumodulator is selected from the list consisting of proteins with SEQ IDs 1-42, peptides with SEQ IDs 1-42, proteins with sequences represented by SEQ IDs 1-42, sequences represented by SEQ IDs 1-42, and protein with any combination of SEQ IDs 1-42.

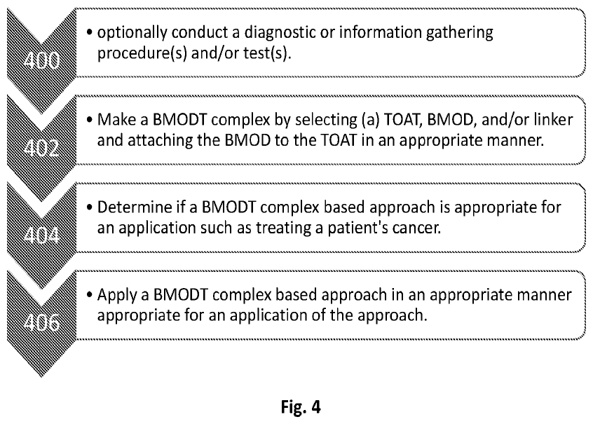

FIG. 4 is a table showing an embodiment of a process that can be used to make BMODT complexes and/or administer BMODT complexes. At 400, optionally conduct a diagnostic or information gathering procedure(s) and/or test(s). This can allow subsequent decisions to be made with more contextual information and help precision medicine, production, and/or engineering. At 402, make a BMODT complex by selecting (a) TOAT, BMOD, and optionally, a linker, and attaching the BMOD to the TOAT in an appropriate manner. The BMOD can be attached using the PEDIP reaction. At 404, determine if a BMODT complex based approach is appropriate for an application such as treating a patient's cancer. At 406, apply a BMODT complex based approach in a manner appropriate for the application. Based on diagnostic or information gathering procedure(s) and/or test(s), the molecules to target with a TOAT and/or BMOD can be determined. For example, if a patient was determined (for example, by biopsy and/or liquid biopsy) to have Her2 positive breast cancer, a TOAT such as an anti-Her2 antibody can be selected and/or a BMOD that can bind to CD16, CD3, and/or Her2 can be selected to make a BMODT complex. The biomarkers for a medical application can be determined or otherwise identified, and based on that information, the appropriate combination of TOAT and BMOD can be selected to make and/or administer an appropriate BMODT complex. A biomarker can be a pattern or an analysis. Applications include basic science research, diagnostic purposes, therapeutic purposes, and other scenarios. The use of the PEDIP reaction to make a BMODT complex and/or attach a BMOD to a TOAT can be novel and very useful. So far, the PEDIP reaction has not been used to attach a scFv, affimer, aptamer, nanobody, binding interface, antibody, antibody derivative, antibody fragment, immunoglobulin, immunoglobulin derivative, immunoglobulin fragment, non-toxin, non-toxic binding interface, and/or BMOD to a TOAT or antibody. Using the PEDIP reaction can be a quick and/or efficient way to make a BMODT complex. Using the PEDIP reaction can be a highly targeted and/or specific way to attach a BMOD to a TOAT and/or to a specific part of the TOAT. Using PEDIP reaction to make a BMODT complex can be a step that can be added at the end or in the later stages of a TOAT production/manufacturing process. Conjugating a BMOD to a TOAT can be a novel process.

In an embodiment, a BMOD can be used to make a BMODT complex that can functionally afucosylate antibodies. This can be because in an embodiment, a BMOD can be used to enhance the binding of an antibody to effector cells such as cells that perform ADCC if a BMOD can bind to effector cells such as cells that perform ADCC in a (enhanced) way. In an embodiment, BMOD sequences can include SEQ IDs 1-42.

In an embodiment, a BMOD can be used to make a BMODT complex that can have a higher (and potentially more diverse) cytotoxic potential than the TOAT it was based on. In an embodiment, a BMOD can be used to make a BMODT complex that can be a biosimilar drug and/or biobetter drug.

In an embodiment, a BMOD can be used to make a BMODT complex that can act like an engager. In an embodiment, a BMOD can be used to make a BMODT complex that can functionally engage (multiple) cell(s). In an embodiment, a BMOD can be used to make a BMODT complex that can functionally achieve a function that a bispecific T-cell engager can achieve. In an embodiment, a BMOD can be used to make a BMODT complex that can be a multispecific cell(s) engager. This BMODT complex can engage and/or activate multiple cells. This BMODT complex can engage target cell(s), immune cell(s), NK cells, dendritic cells, antigen presenting cells, and/or CTLs. For example, a BMODT complex based on a TOAT that can bind to CD19 and a BMOD that can bind to CD3 can achieve a function similar to that of Blinatumomab, potentially with better half-life properties.

A BMODT complex can have a half-life in fluids and/or fluid systems such as blood that is different from the half-life of the TOAT it was based on due to differences in hydrodynamic diameter, surface charges, extent of zwitterionic behavior, binding to other molecules, binding properties, etc.

The foregoing has been a detailed description of illustrative embodiments of the invention. Various modifications and additions can be made without departing from the spirit and scope if this invention. Each of the various embodiments described above may be combined with other described embodiments in order to provide multiple features. Furthermore, while the foregoing describes a number of separate embodiments of the apparatus and method of the present invention, what has been described herein is merely illustrative of the application of the principles of the present invention. For example, in various embodiments, a BMODT complex can include a scFv that is bound to an antibody, or an scFv that is bound to a various other components of the immune system. For example, in various embodiments, a BMOD can be bound to any biologic drug to make a BMODT complex. Also, as used herein, various directional and orientational terms (and grammatical variations thereof) such as “vertical”, “horizontal”, “up”, “down”, “bottom”, “top”, “side”, “front”, “rear”, “left”, “right”, “forward”, “rearward”, and the like, are used only as relative conventions and not as absolute orientations with respect to a fixed coordinate system, such as the acting direction of gravity. Additionally, where the term “substantially” or “approximately” is employed with respect to a given measurement, value or characteristic, it refers to a quantity that is within a normal operating range to achieve desired results, but that includes some variability due to inherent inaccuracy and error within the allowed tolerances (e.g. 1-2%) of the system. Note also, as used herein the terms “process” and/or “processor” should be taken broadly to include a variety of electronic hardware and/or software based functions and components. Moreover, a depicted process or processor can be combined with other processes and/or processors or divided into various sub-processes or processors. Such sub-processes and/or sub-processors can be variously combined according to embodiments herein. Likewise, it is expressly contemplated that any function, process and/or processor herein can be implemented using electronic hardware, software consisting of a non-transitory computer-readable medium of program instructions, or a combination of hardware and software. Accordingly, this description is meant to be taken only by way of example, and not to otherwise limit the scope of this invention.

What is claimed is:

1. A BMOD-TOAT (BMODT) complex comprising:
at least one binding modulator (BMOD); and
at least one of a targeting and a therapeutic agent (TOAT), wherein the at least one BMOD is bound to the at least one TOAT.

2. The BMODT complex of claim 1, wherein the BMOD or a trumodulator is selected from the list consisting of an anti-CD3 scFv, anti-CD16 scFv, nanobody, antibody-based molecule(s), immunoglobulin based molecule(s), BiTe, BiKe, immunoglobulin, antibody, affimer, nucleotide, a synthetic lectin, immunoglobulin binding peptide, Fc binding peptide, albumin binding peptide, Fc binding ligands, FcIII-scFv fusion protein with non-canonical amino acids, FcIII based fusion protein with non-canonical amino acids, FcIII peptide with ncAA fused to a scFv, immunoglobulin based fusion molecule(s), antibody fragment, immunoglobulin fragment, antibody derivative, immunoglobulin fragment, peptides with ncAA(s), fusion proteins that include ncAA(s), binding interfaces, binding interfaces with ncAA(s), fusion protein with ncAA(s), engineered immunoglobulin based fusion molecule(s), protein A, proteins that include one or more of sequence(s) SEQ IDs 1-42, peptides that include one or more of sequence(s) SEQ IDs 1-42, proteins that include one or more of sequence(s) SEQ IDs 1-42, sequences SEQ IDs 1-42, a protein with a combination of one or more of sequence(s) SEQ IDs 1-42, cytokine(s), albumin, zwitterionic amino acids, protein G, a carbohydrate binding molecule, and carbohydrate.

3. The BMODT complex of claim 1, wherein the TOAT is selected from the list consisting of a biologic, immunoglobulin, antibody, affimer, Fab fragment, immunoglobulin(s), molecule that can bind to an epitope, molecule that can bind to an antigen, molecule that can bind to an autoantigen, molecule that can specifically bind to a biologically relevant entity, ligand, IgG, antibiotic, therapy, and component of a therapy.

4. The BMODT complex of claim 2, in which the BMOD is attached to the TOAT with or without a linker.

5. A method of attaching a binding modulator (BMOD) to a therapeutic agent (TOAT) to provide the BMODT complex of claim 1, the method comprising:
mixing the BMOD and the TOAT; and
generating, from the step of mixing, the BMODT.

6. The method of claim 5, wherein blending the BMOD and the TOAT further comprises blending the BMOD and the TOAT in a PEDIP reaction, chemical reaction, photoreactive reaction, non-radiation involved reaction, or a proximity based reaction.

7. The method of claim 6, wherein blending the BMOD and the TOAT in the PEDIP reaction further comprises using a chemical reaction, a photochemical reaction, or a Fc binding peptide.

8. The method of claim 6, further comprising, attaching at least one of a scFv, affimer, aptamer, nanobody, binding interface, antibody, antibody derivative, antibody fragment, immunoglobulin, immunoglobulin derivative, immunoglobulin fragment, non-toxin, non-toxic binding interface, non-toxin binding interface, and the BMOD to the TOAT or to an antibody, using the PEDIP reaction.

9. A method of administering a therapy that includes the at least one BMODT complex of claim 1, comprising the steps of:
collecting biological data from a patient;
analyzing the biological data to determine biomarker(s);
selecting the BMOD or engineered scFv depending on biomarker data;
selecting the TOAT or antibody depending on biomarker data;
sonding the BMOD or engineered scFv to the TOAT/antibody; and infusing the BMODT complex into the patient.

10. The method according to claim 9, wherein a therapy involving the BMODT complex is used to diagnose or treat at least one of cancer, autoimmune disease, rare diseases, inflammatory conditions, medical diseases, and medical conditions.

11. The BMODT complex of claim 2, wherein at least one of the TOAT, the trumodulator, the BMODT, and the BMOD binds to a solid support, semi-solid support, CD3, CD16, PD-L1, CTLA-4, PD1, NKG2D, IL-6, TNF, HER2, CD20, EGFR, IL-17R, IL-12, IL-23, VEGF, GD-2, dabigatran, CD20, BLyS, BAFF, Fc, IgG Fc region, IL-5, PCSK9, PDGFR, C. difficile toxin B, macrophages, immune cells, engineered cells, effector cells, CD33, CD123, CD22, FIXa, FX, IL-5R a subunit, CD47, checkpoint inhibitor, tumor associated antigen, tumor specific antigen, viral protein, autoantigen, microbial protein, human in vitro protein, bacterial protein, IgE, IL-12/23, P-selectin, pathogens, CD4, CD19, RSV, CD52, ITGA4, VEGF-A, C5, IL-1B, TNFa, IL-6R RANKL, BLyS, CD30, B. anthrasis PA, EGFR2, a4B7 integrin, IL-17a, CD38, SLAMF7, IL-23 p19, EpCAM, BAFF, Tau, beta amyloid, amyloid beta, Fc receptor(s), IL-4Ra, Factor IXa, Factor X, FGF23, CCR4, CGRP, IgE Fc region, Fc region, CGRPR, and von Williebrand factor.

12. The BMODT complex of claim 1, wherein the BMODT complex comprises, a super blocker, in which the TOAT is modified or attached with the BMOD that has the same or similar targeting, binding, or specificity properties as the TOAT.

13. The BMODT complex of claim 1, wherein the BMODT complex comprises a vary blocker, in which the TOAT is modified or attached with the BMOD that has targeting, binding, or specificity properties for a target related to, associated with, or in the same class as the target of the TOAT.

14. The BMODT complex of claim 1, wherein the BNODT complex comprises a combo blocker, in which the TOAT is modified or attached to the BMOD that has targeting, binding, or specificity properties for a target that is targeted alongside or in combination with the target of the TOAT.

15. The BMOD of claim 1, wherein the BMOD comprises parts that include a TOAT binder, trumodulator, anti-HAMA, rider, adsorption preventer, and deimmunizer.

16. The BMOD of claim 1, wherein the BMOD comprises an enhancer (EN) system that increases the performance and efficacy of the TOAT and the BMODT complex when the EN system is bound to the TOAT.

17. The EN system of claim 16, further comprising an immunoglobulin engager (IgENG) system that binds to the TOAT, based upon immunoglobulin, and includes IgG and antibodies.

18. The IgENG system of claim 17, wherein the IgENG is used to increase or decrease the at least one of ADCC, TDCC, ADCP, CDC, half-life, exposure, pharmacodynamic properties, pharmacokinetic properties, efficacy, functionality, and cytotoxicity of antibody-based therapies or the immunoglobulin based TOAT.

19. The IgENG system of claim 18, wherein the IgENG system is used to generate the BMODT complex with increased specificity and valency when compared to the TOAT that it is based upon.

20. The BMOD of claim 1, wherein the BMOD comprises a diminisher (DIM) system that is arranged to decrease performance and efficacy of the TOAT and the BMODT complex when the DIM system is bound to the TOAT.


SEQUENCE LISTING (42 sequences)

SEQ ID NO: 1 - protein, 269 aa, Homo Sapiens
WCTGGEFGGGSGGGSQVQLQESGGGLVQPGRSMKLSCAASGFTFSSFPMAWVRQAPKKGLEWVATISTSGGRTYYRDSVKGRFTISRDNGKSILYLQMNSLRSEDTATYYCSRFRQYSGGFDYWGQGTTVTVSSGGGGSGGGGSGGGGSDFMLTQPHSVSESPGKTVIISCTLSSGNIENNYVHWYQQRPGRAPTLVIFDDDKRPDGVPDRFSGSIDRSSNSASLTISGLQTEDEADYYCHSYVSSFNVFGGGTKLTVLHHHHHHHHHH

SEQ ID NO: 2 - protein, 238 aa, Homo Sapiens
  PROPEP (1)..(238)
WCTGGEFGGGSGGGSEVQLVESGGGVVRPGGSLRLSCAASGFTFDDYGMSWVRQAPGKGLEWVSGINWNGGSTGYADSVRFTISRDNAKNSLYLQMNSLRAEDTAVYYCARGRSLLFDYWGQGTLVTVSRSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVG

SEQ ID NO: 3 - protein, 48 aa, Homo sapiens
  PROPEP (1)..(48)
DCAWHLGELPBPAWCTGGEFGGGSGGGSQRLMEDICLPRWGCLWEDDF

SEQ ID NO: 4 - protein, 290 aa, Homo sapiens
  PROPEP (1)..(290)
WCTGGEFGGGSGGGSQVQLQESGGGLVQPGRSMKLSCAASGFTFSSFPMAWVRQAPKKGLEWVATISTSGGRTYYRDSVKGRFTISRDNGKSILYLQMNSLRSEDTATYYCSRFRQYSGGFDYWGQGTTVTVSSGGGGSGGGGSGGGGSDFMLTQPHSVSESPGKTVIISCTLSSGNIENNYVHWYQQRPGRAPTLVIFDDDKRPDGVPDRFSGSIDRSSNSASLTISGLQTEDEADYYCHSYVSSFNVFGGGTKLTVLGGGGSGGGGSGGGGSQRFVTGHFGGLYPANG

SEQ ID NO: 5 - protein, 303 aa, Homo sapiens
  PROPEP (1)..(303)
DCAWHLGELPBPAWCTGGEFGGGSGGGSQVQLQESGGGLVQPGRSMKLSCAASGFTFSSFPMAWVRQAPKKGLEWVATISTSGGRTYYRDSVKGRFTISRDNGKSILYLQMNSLRSEDTATYYCSRFRQYSGGFDYWGQGTTVTVSSGGGGSGGGGSGGGGSDFMLTQPHSVSESPGKTVIISCTLSSGNIENNYVHWYQQRPGRAPTLVIFDDDKRPDGVPDRFSGSIDRSSNSASLTISGLQTEDEADYYCHSYVSSFNVFGGGTKLTVLGGGGSGGGGSGGGGSQRFCTGHFGGLYPCNG

SEQ ID NO: 6 - protein, 303 aa, Homo sapiens
  PROPEP (1)..(303)
DCAWHLGELPBPAWCTGGEFGGGSGGGSQVQLQESGGGLVQPGRSMKLSCAASGFTFSSFPMAWVRQAPKKGLEWVATISTSGGRTYYRDSVKGRFTISRDNGKSILYLQMNSLRSEDTATYYCSRFRQYSGGFDYWGQGTTVTVSSGGGGSGGGGSGGGGSDFMLTQPHSVSESPGKTVIISCTLSSGNIENNYVHWYQQRPGRAPTLVIFDDDKRPDGVPDRFSGSIDRSSNSASLTISGLQTEDEADYYCHSYVSSFNVFGGGTKLTVLGGGGSGGGGSGGGGSQRFVTGHFGGLHPANG

SEQ ID NO: 7 - protein, 290 aa, Homo sapiens
  PROPEP (1)..(290)
WCTGGEFGGGSGGGSQVQLQESGGGLVQPGRSMKLSCAASGFTFSSFPMAWVRQAPKKGLEWVATISTSGGRTYYRDSVKGRFTISRDNGKSILYLQMNSLRSEDTATYYCSRFRQYSGGFDYWGQGTTVTVSSGGGGSGGGGSGGGGSDFMLTQPHSVSESPGKTVIISCTLSSGNIENNYVHWYQQRPGRAPTLVIFDDDKRPDGVPDRFSGSIDRSSNSASLTISGLQTEDEADYYCHSYVSSFNVFGGGTKLTVLGGGGSGGGGSGGGGSQRFCTGHFGGLHPCNG

SEQ ID NO: 8 - protein, 269 aa, Homo sapiens
  PROPEP (1)..(269)
WCTGGEFGGGSGGGSEVQLVESGGGVVRPGGSLRLSCAASGFTFDDYGMSWVRQAPGKGLEWVSGINWNGGSTGYADSVRFTISRDNAKNSLYLQMNSLRAEDTAVYYCARGRSLLFDYWGQGTLVTVSRSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVGGGGGSGGGGSGGGGSQRFVTGHFGGLYPANG

SEQ ID NO: 9 - protein, 268 aa, Homo sapiens
WCTGGEFGGGSGGGSEVQLVESGGGVVRPGGSLRLSCAASGFTFDDYGMSWVRQAPGKGLEWVSGINWNGGSTGYADSVRFTISRDNAKNSLYLQMNSLRAEDTAVYYCARGRSLLFDYWGQGTLVTVSRSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVGGGGSGGGGSGGGGSQRFCTGHFGGLYPCNG

SEQ ID NO: 10 - protein, 268 aa, Homo sapiens
  PROPEP (1)..(268)
WCTGGEFGGGSGGGSEVQLVESGGGVVRPGGSLRLSCAASGFTFDDYGMSWVRQAPGKGLEWVSGINWNGGSTGYADSVRFTISRDNAKNSLYLQMNSLRAEDTAVYYCARGRSLLFDYWGQGTLVTVSRSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVGGGGSGGGGSGGGGSQRFVTGHFGGLHPANG

SEQ ID NO: 11 - protein, 268 aa, Homo sapiens
  PROPEP (1)..(268)
WCTGGEFGGGSGGGSEVQLVESGGGVVRPGGSLRLSCAASGFTFDDYGMSWVRQAPGKGLEWVSGINWNGGSTGYADSVRFTISRDNAKNSLYLQMNSLRAEDTAVYYCARGRSLLFDYWGQGTLVTVSRSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVGGGGSGGGGSGGGGSQRFCTGHFGGLHPCNG

SEQ ID NO: 12 - protein, 66 aa, Homo sapiens
  PROPEP (1)..(66)
WCTGGEFGGGSGGGSQRLMEDICLPRWGCLWEDDFGGGGSGGGGSGGGGSQRFVTGHFGGLYPANG

SEQ ID NO: 13 - protein, 66 aa, Homo sapiens
  PROPEP (1)..(66)
WCTGGEFGGGSGGGSQRLMEDICLPRWGCLWEDDFGGGGSGGGGSGGGGSQRFCTGHFGGLYPCNG

SEQ ID NO: 14 - protein, 66 aa, Homo sapiens
  PROPEP (1)..(66)
WCTGGEFGGGSGGGSQRLMEDICLPRWGCLWEDDFGGGGSGGGGSGGGGSQRFVTGHFGGLHPANG

SEQ ID NO: 15 - protein, 66 aa, Homo sapiens
  PROPEP (1)..(66)
WCTGGEFGGGSGGGSQRLMEDICLPRWGCLWEDDFGGGGSGGGGSGGGGSQRFCTGHFGGLHPCNG

SEQ ID NO: 16 - protein, 318 aa, Homo sapiens
  PROPEP (1)..(318)
WCTGGEFGGGSGGGSQVQLQESGGGLVQPGRSMKLSCAASGFTFSSFPMAWVRQAPKKGLEWVATISTSGGRTYYRDSVKGRFTISRDNGKSILYLQMNSLRSEDTATYYCSRFRQYSGGFDYWGQGTTVTVSSGGGGSGGGGSGGGGSDFMLTQPHSVSESPGKTVIISCTLSSGNIENNYVHWYQQRPGRAPTLVIFDDDKRPDGVPDRFSGSIDRSSNSASLTISGLQTEDEADYYCHSYVSSFNVFGGGTKLTVLGGGSGGGSQRLMEDICLPRWGCLWEDDFGGGGSGGGGSGGGGSQRFVTGHFGGLYPANG

SEQ ID NO: 17 - protein, 318 aa, Homo sapiens
  PROPEP (1)..(318)
WCTGGEFGGGSGGGSQVQLQESGGGLVQPGRSMKLSCAASGFTFSSFPMAWVRQAPKKGLEWVATISTSGGRTYYRDSVKGRFTISRDNGKSILYLQMNSLRSEDTATYYCSRFRQYSGGFDYWGQGTTVTVSSGGGGSGGGGSGGGGSDFMLTQPHSVSESPGKTVIISCTLSSGNIENNYVHWYQQRPGRAPTLVIFDDDKRPDGVPDRFSGSIDRSSNSASLTISGLQTEDEADYYCHSYVSSFNVFGGGTKLTVLGGGSGGGSQRLMEDICLPRWGCLWEDDFGGGGSGGGGSGGGGSQRFCTGHFGGLYPCNG

SEQ ID NO: 18 - protein, 318 aa, Homo sapiens
  PROPEP (1)..(318)
WCTGGEFGGGSGGGSQVQLQESGGGLVQPGRSMKLSCAASGFTFSSFPMAWVRQAPKKGLEWVATISTSGGRTYYRDSVKGRFTISRDNGKSILYLQMNSLRSEDTATYYCSRFRQYSGGFDYWGQGTTVTVSSGGGGSGGGGSGGGGSDFMLTQPHSVSESPGKTVIISCTLSSGNIENNYVHWYQQRPGRAPTLVIFDDDKRPDGVPDRFSGSIDRSSNSASLTISGLQTEDEADYYCHSYVSSFNVFGGGTKLTVLGGGSGGGSQRLMEDICLPRWGCLWEDDFGGGGSGGGGSGGGGSQRFVTGHFGGLHPANG

SEQ ID NO: 19 - protein, 318 aa, Homo sapiens
  PROPEP (1)..(318)
WCTGGEFGGGSGGGSQVQLQESGGGLVQPGRSMKLSCAASGFTFSSFPMAWVRQAPKKGLEWVATISTSGGRTYYRDSVKGRFTISRDNGKSILYLQMNSLRSEDTATYYCSRFRQYSGGFDYWGQGTTVTVSSGGGGSGGGGSGGGGSDFMLTQPHSVSESPGKTVIISCTLSSGNIENNYVHWYQQRPGRAPTLVIFDDDKRPDGVPDRFSGSIDRSSNSASLTISGLQTEDEADYYCHSYVSSFNVFGGGTKLTVLGGGSGGGSQRLMEDICLPRWGCLWEDDFGGGGSGGGGSGGGGSQRFCTGHFGGLHPCNG

SEQ ID NO: 20 - protein, 297 aa, Homo sapiens
  PROPEP (1)..(297)
WCTGGEFGGGSGGGSEVQLVESGGGVVRPGGSLRLSCAASGFTFDDYGMSWVRQAPGKGLEWVSGINWNGGSTGYADSVRFTISRDNAKNSLYLQMNSLRAEDTAVYYCARGRSLLFDYWGQGTLVTVSRSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVGGGGSGGGSQRLMEDICLPRWGCLWEDDFGGGGSGGGGSGGGGSQRFVTGHFGGLYPANG

SEQ ID NO: 21 - protein, 297 aa, Homo sapiens
  PROPEP (1)..(297)
WCTGGEFGGGSGGGSEVQLVESGGGVVRPGGSLRLSCAASGFTFDDYGMSWVRQAPGKGLEWVSGINWNGGSTGYADSVRFTISRDNAKNSLYLQMNSLRAEDTAVYYCARGRSLLFDYWGQGTLVTVSRSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVGGGGSGGGSQRLMEDICLPRWGCLWEDDFGGGGSGGGGSGGGGSQRFCTGHFGGLYPCNG

SEQ ID NO: 22 - protein, 297 aa, Homo sapiens
  PROPEP (1)..(297)
WCTGGEFGGGSGGGSEVQLVESGGGVVRPGGSLRLSCAASGFTFDDYGMSWVRQAPGKGLEWVSGINWNGGSTGYADSVRFTISRDNAKNSLYLQMNSLRAEDTAVYYCARGRSLLFDYWGQGTLVTVSRSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVGGGGSGGGSQRLMEDICLPRWGCLWEDDFGGGGSGGGGSGGGGSQRFVTGHFGGLHPANG

SEQ ID NO: 23 - protein, 297 aa, Homo sapiens
  PROPEP (1)..(297)
WCTGGEFGGGSGGGSEVQLVESGGGVVRPGGSLRLSCAASGFTFDDYGMSWVRQAPGKGLEWVSGINWNGGSTGYADSVRFTISRDNAKNSLYLQMNSLRAEDTAVYYCARGRSLLFDYWGQGTLVTVSRSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVGGGGSGGGSQRLMEDICLPRWGCLWEDDFGGGGSGGGGSGGGGSQRFCTGHFGGLHPCNG

SEQ ID NO: 24 - protein, 259 aa, Homo sapiens
  PROPEP (1)..(259)
WCTGGEFGGGSGGGSQVQLQESGGGLVQPGRSMKLSCAASGFTFSSFPMAWVRQAPKKGLEWVATISTSGGRTYYRDSVKGRFTISRDNGKSILYLQMNSLRSEDTATYYCSRFRQYSGGFDYWGQGTTVTVSSGGGGSGGGGSGGGGSDFMLTQPHSVSESPGKTVIISCTLSSGNIENNYVHWYQQRPGRAPTLVIFDDDKRPDGVPDRFSGSIDRSSNSASLTISGLQTEDEADYYCHSYVSSFNVFGGGTKLTVL

SEQ ID NO: 25 - protein, 262 aa, Homo sapiens
  PROPEP (1)..(262)
WCTGGGGSGGGGSGGGGSQVQLQESGGGLVQPGRSMKLSCAASGFTFSSFPMAWVRQAPKKGLEWVATISTSGGRTYYRDSVKGRFTISRDNGKSILYLQMNSLRSEDTATYYCSRFRQYSGGFDYWGQGTTVTVSSGGGGSGGGGSGGGGSDFMLTQPHSVSESPGKTVIISCTLSSGNIENNYVHWYQQRPGRAPTLVIFDDDKRPDGVPDRFSGSIDRSSNSASLTISGLQTEDEADYYCHSYVSSFNVFGGGTKLTVL

SEQ ID NO: 26 - protein, 267 aa, Homo sapiens
  PROPEP (1)..(267)
WCTGGGGSGGGGSGGGGSGGGGSQVQLQESGGGLVQPGRSMKLSCAASGFTFSSFPMAWVRQAPKKGLEWVATISTSGGRTYYRDSVKGRFTISRDNGKSILYLQMNSLRSEDTATYYCSRFRQYSGGFDYWGQGTTVTVSSGGGGSGGGGSGGGGSDFMLTQPHSVSESPGKTVIISCTLSSGNIENNYVHWYQQRPGRAPTLVIFDDDKRPDGVPDRFSGSIDRSSNSASLTISGLQTEDEADYYCHSYVSSFNVFGGGTKLTVL

SEQ ID NO: 27 - protein, 241 aa, Homo sapiens
  PROPEP (1)..(241)
WCTGGGGSGGGGSGGGGSEVQLVESGGGVVRPGGSLRLSCAASGFTFDDYGMSWVRQAPGKGLEWVSGINWNGGSTGYADSVRFTISRDNAKNSLYLQMNSLRAEDTAVYYCARGRSLLFDYWGQGTLVTVSRSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVG

SEQ ID NO: 28 - protein, 246 aa, Homo sapiens
  PROPEP (1)..(246)
WCTGGGGSGGGGSGGGGSGGGGSEVQLVESGGGVVRPGGSLRLSCAASGFTFDDYGMSWVRQAPGKGLEWVSGINWNGGSTGYADSVRFTISRDNAKNSLYLQMNSLRAEDTAVYYCARGRSLLFDYWGQGTLVTVSRSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVG

SEQ ID NO: 29 - protein, 38 aa, Homo sapiens
  PROPEP (1)..(38)
WCTGGGGSGGGGSGGGGSQRLMEDICLPRWGCLWEDDF

SEQ ID NO: 30 - protein, 43 aa, Homo sapiens
  PROPEP (1)..(43)
WCTGGGGSGGGGSGGGGSGGGGSQRLMEDICLPRWGCLWEDDF

SEQ ID NO: 31 - protein, 74 aa, Homo sapiens
  PROPEP (1)..(74)
WCTGGGGSGGGGSGGGGSGGGGSQRLMEDICLPRWGCLWEDDFGGGGSGGGGSGGGGSQRFVTGHFGGLYPANG

SEQ ID NO: 32 - protein, 74 aa, Homo sapiens
  PROPEP (1)..(74)
WCTGGGGSGGGGSGGGGSGGGGSQRLMEDICLPRWGCLWEDDFGGGGSGGGGSGGGGSQRFCTGHFGGLYPCNG

SEQ ID NO: 33 - protein, 74 aa, Homo sapiens
  PROPEP (1)..(74)
WCTGGGGSGGGGSGGGGSGGGGSQRLMEDICLPRWGCLWEDDFGGGGSGGGGSGGGGSQRFVTGHFGGLHPANG

SEQ ID NO: 34 - protein, 74 aa, Homo sapiens
  PROPEP (1)..(74)
WCTGGGGSGGGGSGGGGSGGGGSQRLMEDICLPRWGCLWEDDFGGGGSGGGGSGGGGSQRFCTGHFGGLHPCNG

SEQ ID NO: 35 - protein, 259 aa, Homo sapiens; identical to SEQ ID NO: 24
  PROPEP (1)..(259)

SEQ ID NO: 36 - protein, 16 aa, Homo sapiens
  PROPEP (1)..(16)
QRFVTGHFGGLYPANG

SEQ ID NO: 37 - protein, 16 aa, Homo sapiens
  PROPEP (1)..(16)
QRFCTGHFGGLYPCNG

SEQ ID NO: 38 - protein, 16 aa, Homo sapiens
  PROPEP (1)..(16)
QRFVTGHFGGLHPANG

SEQ ID NO: 39 - protein, 16 aa, Homo sapiens
  PROPEP (1)..(16)
QRFCTGHFGGLHPCNG

SEQ ID NO: 40 - protein, 20 aa, Homo sapiens
  PROPEP (1)..(20)
QRLMEDICLPRWGCLWEDDF

SEQ ID NO: 41 - protein, 244 aa, Homo sapiens
  PROPEP (1)..(244)
QVQLQESGGGLVQPGRSMKLSCAASGFTFSSFPMAWVRQAPKKGLEWVATISTSGGRTYYRDSVKGRFTISRDNGKSILYLQMNSLRSEDTATYYCSRFRQYSGGFDYWGQGTTVTVSSGGGGSGGGGSGGGGSDFMLTQPHSVSESPGKTVIISCTLSSGNIENNYVHWYQQRPGRAPTLVIFDDDKRPDGVPDRFSGSIDRSSNSASLTISGLQTEDEADYYCHSYVSSFNVFGGGTKLTVL

SEQ ID NO: 42 - protein, 223 aa, Homo sapiens
  PROPEP (1)..(223)
EVQLVESGGGVVRPGGSLRLSCAASGFTFDDYGMSWVRQAPGKGLEWVSGINWNGGSTGYADSVRFTISRDNAKNSLYLQMNSLRAEDTAVYYCARGRSLLFDYWGQGTLVTVSRSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVG